(19) United States
(12) Patent Application Publication
(10) Pub. No.: US 2005/0123587 A1
(43) Pub. Date: Jun. 9, 2005

(54) Fluid composition for increased total water intake

(75) Inventors: Shiguang Yu, Topeka, KS (US); Charles Lin, Walnut, CA (US); Claudia Ann Kirk, Louisville, TN (US)

Correspondence Address: HARNESS, DICKEY & PIERCE, P.L.C., P.O. BOX 8910, RESTON, VA, 20195, US

(73) Assignee: Hill's Pet Nutrition, Inc., Topeka, KS (US)

(21) Appl. No.: 10/979,760

(22) Filed: Nov. 3, 2004

Related U.S. Application Data
(60) Provisional application No. 60/518,555, filed on Nov. 7, 2003.

(51) Int. Cl. A61K 35/407; A23K 1/165

(52) U.S. Cl. 424/442; 424/553

(57) ABSTRACT

A highly palatable fluid composition for oral consumption is provided which contains liver digest and results in increased total water intake and urine production and decreased urinary specific gravity in companion animals. Methods of using these compositions for providing increased total water intake in companion animals are also provided.

### CROSS-REFERENCE TO RELATED APPLICATION

This application is a non-provisional of U.S. Provisional Application No. 60/518,555, filed on Nov. 7, 2003, the contents of which are incorporated herein by reference in their entirety for all purposes.

### FIELD OF THE INVENTION

The invention provides palatable fluid compositions which result in increased total water intake.

### BACKGROUND

Dogs and cats suffer from numerous disorders of the lower urinary tract. Among these are idiopathic urinary tract disease, crystalluria, bacterial cystitis, urolithiasis, idiopathic obstruction, urethral plugs, and the like. A specific disorder is Lower Urinary Tract Disease (LUTD), which is common to cats. Urolithiasis, i.e., stone formation in the urinary tract, is a condition commonly found in both dogs and cats. Although the etiology of these disorders is not completely clear, at least some of the factors associated with these disorders appear to be concentrated urine, i.e., urine having high specific gravity or high mineral supersaturation of urine. Lowering mineral concentrations in the urine by increasing urine production through increased water consumption can reduce the risk of urinary crystal or stone formation, assist in dissolving certain types of formed urinary tract stones, and reduce the occurrence of feline LUTD. Cats are particularly thirst tolerant and often produce highly concentrated urine, making them especially vulnerable to urolithiasis. In addition, increased urine volume initiates more frequent voiding, which further reduces the risk of urinary tract infection, crystalluria, and urolithiasis.

Increased total water intake advantageously results in improved rates of urine production from a companion animal. A fluid that induces or facilitates increased total water intake may be used, for example, to prevent and/or treat lower urinary tract disorder(s) including crystalluria, urolithiasis, cystitis, idiopathic obstruction, urethral plugs, and feline LUTD. In addition, increased total water intake improves hydration and generally benefits animals having conditions including diabetes, renal disease, pregnancy, lactation, and the like.

A problem exists in that there is no commercially available product of which we are aware that may be used to treat companion animals in need of increased total water intake, wherein said product induces voluntary water intake.

Thus, it would be a significant contribution to the art to provide a fluid composition that is highly palatable, and which also results in increased fluid intake, especially where the companion animal is a dog or a cat.

### SUMMARY

The present composition provides a fluid composition containing a flavor enhancer, such as liver digest, for ingestion by a companion animal. The composition is highly palatable and results in increased water intake.

The composition may be used according to a method for feeding a companion animal in need of increased fluid intake.

The composition may be provided in conjunction with additional materials that provide a balanced overall diet meeting the nutritional needs of a dog or cat.

Still further provided is a method of providing the fluid to a dog or cat for the purpose of at least assisting in preventing lower urinary tract disorder(s) including crystalluria, urolithiasis, cystitis, LUTD, idiopathic obstruction, urethral plugs, and the like, in the dog or cat.

Also provided is a method of treating a dog or cat with lower urinary tract disorders which comprises providing the fluid of the invention to a dog or cat having such disorders.

### DETAILED DESCRIPTION

Management of fluid intake in companion animals can be a significant problem, particularly for those companion animals with urinary tract disease or those animals that are at risk of developing urinary tract diseases.

The present composition contains a flavor enhancing agent, such as liver digest, that is useful in increasing fluid intake in companion animals, and particularly for use in dogs or cats. The liver digest is mixed with water, as described below, and one example is a fluid composition referred to as SAWA fluid.

Unless otherwise specified, the term “wt %,” as employed throughout the specification and claims, refers to weight percentage of the total composition calculated on a w/w basis in the aqueous or liquid phase. This definition recognizes, for example, that solids introduced to a mixture may not all dissolve into an aqueous phase, that the aqueous phase may include a gel, and that the aqueous phase may include a suspension, emulsion, or solution of the protein digest.

The liver in the liver digest may be from any source. A liver source that is readily available and easy to prepare is particularly preferred. The liver in the composition may be chemically or enzymatically treated or digested. Liver digest is also commercially available. Examples of such liver sources include pork, beef, chicken, and mixtures thereof. Preferred for the practice of the present invention is pork or chicken liver.

Liver digest concentrations provided in the present composition are suitably from about 1 to about 20 wt %. Suitable representative minimum liver digest concentrations include, for example, 1, 5, 10, and 15 wt %. Suitable representative maximum liver digest concentrations include 10 and 20 wt %. Preferred for the present invention are liver digest concentrations of from about 3 to about 10 wt %. Particularly preferred for the practice of the present invention are liver digest concentrations of from about 3 to about 7 wt %. Particularly preferred for the practice of the present invention is a food composition containing liver digest concentrations of about 5 wt %. In one aspect, the concentration of the liver digest may be a minimum concentration that achieves improved palatability, as indicated by observing evidence of the companion animal making a voluntary preference to choose the composition over plain water.

When the term “food” is used, this may refer not only to a food product which typically provides most, if not all, the nutrient value for a companion animal, but may also refer to such items as a snack, treat, supplement, and the like.

The composition may be provided to any mammal, particularly a companion animal, such as a dog or cat, which is in need of increased fluid intake. For example, the animal, especially a dog or cat, may appear to be healthy, or one which currently has lower urinary tract disease (LUTD) or one which has a tendency to develop LUTD. Temporarily, the animal may require increased fluid intake as a result of trauma to the animal, such as illness, injury, or surgery, and the like.

The desired effect provided by the present fluid composition has been observed in the dog or cat as an increase in overall fluid consumption upon being provided the present fluid composition as a fluid source accompanied by increased excretion. These observations may be made in animals which are already dehydrated, where the feeding of the fluid composition is intended to alleviate or reduce this problem.

The usage of the fluid composition brings about a much higher total water intake for dogs and cats, a higher urine output, and a correspondingly lower urinary specific gravity for them compared to without the fluid composition. In addition, it provides pet owners with a fluid treat of very low energy content. This is particularly useful for pets such as cats and dogs with problems relating to being overweight or of obesity, or other conditions such as arthritis, diabetes, hypertension, and cardiovascular diseases. A highly palatable fluid treat also improves water balance in animals having high protein diets and/or conditions that increase water intake needs, such as diabetes, lactation, exercise, and growth.

Additional palatability enhancers or flavorants may be employed in addition to the liver digest for further enhancement of the palatability to the cat or dog. There are numerous such materials available including animal digests; animal hydrolysates; animal internal organs and digests thereof, such as lungs and heart; meats, such as beef, lamb, pork, chicken, and turkey; sea foods, such as fish, crab, and shrimp; dairy products, such as milk, cheese, or whey concentrates; yeast; peptides; amino acids; nucleotides; fats; oils; artificial meat and/or sea food flavors; maillard reactants; sugars; plant extracts; and aromatic materials, including natural and/or artificial materials that are attractive to cats or dogs.

The amount of the additional palatability enhancer is sufficient to bring about a palatability enhancement, as may be shown by observing the companion animal making a voluntary choice in the preference of the composition that contains these materials over water. This is generally between about 0.1 and 10 wt %, and desirably a minimum wt % of about 0.3 or 1.0 wt %.

Other components can be included, for example, nutrients such as vitamins and minerals used as dietary supplements; preservatives; colorant(s); active agents including antibacterial agent(s), anti-inflammatory agent(s), and antiparasitic(s); antioxidant(s); herbal and/or botanical extracts; thickeners and the like, all provided in effective quantities. The amount of the additional components is sufficient to bring about a desired effect. This is generally between about 0.01 and 5.0 wt. %, and desirably a minimum wt % of about 0.1 or 1.0 wt. % of the fluid.

In one aspect, the other components may additionally include dietary supplements, such as a predetermined amount of amino acids, protein, vitamins, or minerals, that are generally recognized in the art as providing nutritional support for good animal health. In still other aspects the supplements may contain specific formulations recognized by veterinarians as providing nutritional support for prophylactic maintenance of animals that are at increased risk of encountering a disease or condition, such as diabetes or obesity in older cats. In other aspects the supplements may include antibiotics or other drugs that may be consumed orally and which are unpalatable to the companion animal in pure form.

Thus, the fluid can function as a delivery system for supplements as well as active ingredients.

The composition is readily prepared and mixed by standard methods. For example, the fluid may be prepared by mixing all of the components into a container and stirring under conventional or elevated temperatures whenever appropriate. Solids may be removed by filtration or centrifugation before or after the components are mixed.

Overall, dogs and cats can experience significant increases in total water intake and urine production as well as a significant decrease in urine specific gravity when utilizing the fluid of this invention with a diet meeting nutritional requirements. The water intake of dogs and cats is primarily supplemented by water in the environments where most of these companion animals live. When a dry or canned food diet is supplemented with the instant fluid composition, the dog or cat can experience an increase in total water intake of at least about 20%, and this may increase to 70% or 80% of prior levels by weight. Urine production may increase by at least about 20%, and improvements may be obtained of more than about 90% or 100% by weight of prior levels. Urine specific gravity can decrease by at least about 20%, 30% or desirably at least about 40% by weight in comparison to prior levels.

### EXAMPLES

The effect on cats and dogs provided by the SAWA fluid composition described above was assessed by the nonlimiting examples that follow.

### Example 1

### Sawa Fluid Preparation

Frozen pork liver was ground through a ⅛″ plate. A 200 lb portion of the ground pork liver was placed into a kettle having a 300 lb capacity. A 20 lb quantity of water was added to the kettle. The mixture was heated to 145° F. with mixing by paddle. A 181.6 g quantity of ENZECO AS-L, a commercially available fungal protease sold by Enzyme Development Co., was added to the mixture. The mixture was then held at 145° F. for 1 hour, followed by heating to 160° F. A 181.6 g quantity of Papain 6000, a commercially available plant protease sold by Valley Research, Inc., was added. The mixture was heated to 195° F., and held at that temperature for 15 minutes. The mixture was then cooled to 100° F., and filtered through a #20 screen (US mesh). The portion that passed through the screen was collected for use as a “palatability enhancer” fraction. The palatability enhancer was then added to deionized water forming an aqueous mixture that contained 5% by weight of the palatability enhancer. The deionized water and palatability enhancer were combined in an APV mixing container, and mixed for 30 minutes. The mixture was allowed to gravity-separate overnight while running cold tap water through the jacket of the APV container. The supernatant was decanted from the APV and used to fill a plurality of 14 oz. cans. The cans were sealed, and retorted at 252° F. for 45 minutes. The cans were cooled to room temperature and identified as SAWA.

### Example 2

### Cat Study

The SAWA that was prepared in Example 1 was tested in eight adult cats fed a commercial dry maintenance cat food. Total water intake, total urine output, and urine specific gravity and others were measured for each study group. A crossover study design was used. Cats were randomly divided into two groups with four cats in each group. One group was given drinking water only provided in two plastic bottles, each of which contained about 500 grams of drinking water (control group). The other group was given drinking water and SAWA provided in two plastic bottles, one contained about 500 grams of drinking water and the other contained about 500 grams of SAWA (SAWA group). After two weeks, the control and the SAWA groups were crossed over and treated accordingly for another two weeks. Water intake and urine output measurements were performed daily during weeks two and four of the study. Total water intake was measured as the sum of water intake from drinking water and water contained in the food for the control group, and the sum of water intake from drinking water and water in SAWA and the food for the SAWA group. Total urine output was measured by capturing urine discharge from individual cats in the study groups using a capped plastic bottle that was connected with a plastic tube to the stainless steel pan where the cat was housed, and measured by the difference in weight of the plastic bottle before and after the urine collection. Urine specific gravity was measured with a refractometer. Results are shown as averages for the study groups. Major minerals in the urine and urine pH were also measured. Urinary mineral concentration was measured using an optical emission spectrometer (Optima 4300 DV, PerkinElmer Instruments, Norwalk, Conn., USA) and urine pH was measured with a pH meter. To assess relative palatability, drinking water was made available in addition to SAWA during the study and the respective weight of consumption was recorded. The cats drank almost exclusively of SAWA.

Table 1 shows that the SAWA significantly increased total water intake and urine production, and decreased urine specific gravity. Urine mineral concentrations of phosphorus, magnesium, calcium, sodium, and potassium were significantly reduced. Urine pH was not affected by the SAWA. No adverse effects were observed during the study. These results demonstrated that SAWA is useful in the prevention, treatment and management of urolithiasis in cats by increasing urine volume and diluting urinary mineral concentration.

TABLE 1
CAT STUDY RESULTS¹

| Treatments | Water | SAWA | p value² |
|---|---|---|---|
| SAWA intake (g/day) | | 125.3 | |
| Water intake (g/day) | 71.5 | 5.7 | <0.01 |
| Total water intake (g/day)³ | 75.0 | 133.8 | <0.01 |
| Urine production (g/day) | 39.5 | 89.8 | <0.01 |
| Urine specific gravity | 1.059 | 1.029 | <0.01 |
| Urine phosphorus (ppm) | 2717 | 1390 | <0.01 |
| Urine magnesium (ppm) | 92.8 | 57.0 | <0.01 |
| Urine calcium (ppm) | 39.5 | 18.9 | <0.01 |
| Urine sodium (ppm) | 2857 | 1248 | <0.01 |
| Urine potassium (ppm) | 6955 | 3417 | 0.019 |
| Urine pH | 6.67 | 6.73 | 0.611 |
| Fecal score | 4.8 | 4.9 | 0.696 |
| Body weight change (%)⁴ | −0.26 | −0.01 | 0.748 |
| Food intake (g/day) | 45.1 | 45.7 | 0.687 |
| Dry matter intake (g/day) | 41.6 | 42.9 | 0.278 |

¹Average for eight cats
²Paired t-test
³Include water from deionized water, food, and SAWA
⁴% of body weight at the beginning of the test

### Example 3

### Cat Study Using Comparative Products

This study compared the efficacy of the SAWA from Example 1 with commercially available products that are formulated and marketed as pet drinks. SAWA was compared against commercially available materials, including Waltham® Feline/Canine Veterinary Diet™ Rehydration Support™, and tuna juice from canned StarKist® chunk light tuna in water on water balance. The tuna juice was diluted with water so that nitrogen concentration in diluted tuna juice was similar to that in the SAWA.

A similar crossover study design as in Example 2 was used. Instead of 2 groups in Example 2, four groups were tested. They are control (drinking water), SAWA, Waltham® Feline/Canine Veterinary Diet™ Rehydration Support™, and diluted tuna juice. Eight cats were fed maintenance dry cat food. The test methodology followed the methodology of Example 2, substituting each of the products for the SAWA of Example 2. The study results are presented in Table 2.

SAWA, as observed in Example 2, significantly increased total water intake and urine volume and decreased urinary specific gravity. Urinary mineral concentrations of phosphorus, magnesium, calcium, sodium, and potassium were also significantly reduced.

Waltham® Feline/Canine Veterinary Diet™ Rehydration Support™ is a veterinary product that is formulated with electrolytes to facilitate rehydration after acute diarrhea, vomiting, and veterinary surgery. The product contains maltodextrin, sodium, potassium, citrate and glycine at ratios and concentrations regarded by veterinarians as being acceptable and effective in compensating for water and electrolyte losses and for restoring acid-base balance in dehydrated dogs and cats. This test group did not experience increased total water intake (when corrected with dry matter intake) nor increased urine production, and the product had little or no effect on water balance in cats when compared with the cats in the control group but significantly increased sodium excretion in the urine, which can be detrimental in cats with chronic renal failure.

The diluted tuna juice contained juice that was decanted from StarKist® brand light tuna packed in water, and was diluted ten times using tap water so that nitrogen concentration in diluted tuna juice was similar to that in the SAWA.

TABLE 2
RESULTS¹ OF CAT STUDY USING COMPARATIVE PRODUCTS

| | Water source | | | | p value⁴ | | |
|---|---|---|---|---|---|---|---|
| Treatments | Water | SAWA | WRS² | Tuna Juice³ | SAWA | WRS | Tuna Juice³ |
| Pet drink intake (g/day) | | 99.6 | 69.9 | 120.8 | | | |
| Water intake (g/day) | 69.6 | 4.9 | 12.1 | 4.4 | | | |
| Total water intake (g/day)⁵ | 73.6 | 107.8 | 81.9 | 128.3 | 0.002 | 0.014 | 0.002 |
| Total water intake (g/g DM)⁵ | 1.6 | 2.3 | 1.7 | 2.8 | 0.002 | 0.172 | 0.001 |
| Urine production (g/day) | 39.1 | 68.6 | 43 | 88.3 | 0.004 | 0.088 | 0.002 |
| Urine specific gravity | 1.059 | 1.040 | 1.053 | 1.028 | 0.001 | 0.005 | <0.01 |
| Urine pH | 7.14 | 6.93 | 6.96 | 6.86 | 0.178 | 0.208 | 0.137 |
| Urine phosphorus (ppm) | 2076 | 1460 | 1794 | 1145 | 0.003 | 0.069 | <0.01 |
| Urine magnesium (ppm) | 77.1 | 56.0 | 66.6 | 50.3 | 0.001 | 0.129 | 0.014 |
| Urine calcium (ppm) | 36.4 | 26.3 | 36.4 | 23.3 | 0.005 | 0.500 | 0.002 |
| Urine sodium (ppm) | 3309.4 | 2215 | 4160 | 2063 | 0.001 | <0.01 | 0.001 |
| Urine potassium (ppm) | 7637 | 5043 | 7524 | 4006 | 0.001 | 0.289 | <0.001 |
| Fecal score | 4.8 | 4.8 | 4.7 | 4.8 | 0.316 | 0.327 | 0.356 |
| Body weight change (%)⁶ | 0.08 | 0.00 | 0.33 | −0.39 | 0.829 | 0.750 | 0.376 |
| Food intake (g/day) | 49.5 | 50 | 49.1 | 48.8 | 0.148 | 0.093 | 0.234 |
| Dry matter intake (g/day) | 45.5 | 46.6 | 49.2 | 45.6 | 0.017 | <0.01 | 0.462 |

¹Average for seven cats
²Waltham Feline/Canine Veterinary Diet ™ Rehydration Support ™
³Tuna Juice from StarKist chunk light tuna in water and diluted 10 times with tap water
⁴Paired t-test, comparing with water
⁵Include water from deionized water, food, and pet drinks
⁶% of body weight at the beginning of the test

TABLE 3
COMPARISON BETWEEN SAWA, TUNA JUICE, AND PET DRINKS

| Compositions | SAWA (%) | Tuna Juice (%)¹ | WRS² (g/100 ml) |
|---|---|---|---|
| Moisture | 99.58 | 94.31 | 94.5 |
| Protein | <1 | 4.5 | 1.1 |
| Fat | <0.1 | <0.1 | 0 |
| Ash | 0.05 | 1.06 | 1.3 |
| Crud fiber | <0.2 | <0.01 | 0 |
| NFE | <0.1 | <0.1 | 19 |
| Sodium | 0.005 | 0.2 | 0.4 |
| Potassium | 0.008 | 0.2 | 0.3 |
| Phosphorus | <0.005 | 0.12 | — |
| Calcium | <0.005 | <0.01 | — |
| Magnesium | <0.0002 | <0.01 | — |

¹Undiluted tuna juice decanted from StarKist ® brand light tuna packed in water
²Waltham ® Feline/Canine Veterinary Diet ™ Rehydration Support ™

In addition, cats drank almost exclusively from the SAWA fluid bottle even though water was available. Further, the other parameters measured, i.e., body weight, food intake, urine pH, and stool quality, were not affected by the SAWA fluid.

### Example 4

### Dog Study

SAWA prepared according to Example 1 was tested in eight adult dogs fed a dry commercial maintenance food. The test methodology was the same as methodology used in Example 2, except the test population consisted of eight adult beagles and plastic water bottles were replaced with plastic bowls. Table 4 presents the study results.

The SAWA significantly increased total water intake and urine production, and decreased urine specific gravity. Urine mineral concentration was reduced. These results demonstrate that SAWA could be useful in the prevention and management of urolithiasis in dogs by increasing urine volume and decreasing urine specific gravity.

TABLE 4
DOG STUDY RESULTS¹

| Treatments | Water | SAWA | p value² |
|---|---|---|---|
| SAWA intake (g/day) | | 610 | |
| Water intake (g/day) | 538 | 306 | 0.030 |
| Total water intake (g/day)³ | 555 | 930 | <0.01 |
| Total water intake (g/g DM)³ | 2.8 | 4.6 | 0.009 |
| Urine production (g/day) | 248 | 528 | 0.011 |
| Urine specific gravity | 1.027 | 1.018 | 0.024 |
| Urine phosphorus (ppm) | 1239 | 970 | 0.129 |
| Urine magnesium (ppm) | 57 | 46 | 0.143 |
| Urine calcium (ppm) | 35 | 30 | 0.392 |
| Urine sodium (ppm) | 1709 | 1251 | 0.135 |
| Urine potassium (ppm) | 4337 | 3101 | 0.078 |
| Urine Ph | 8.27 | 8.14 | 0.861 |
| Fecal score | 4.58 | 4.56 | 0.861 |
| Body weight change (%)⁴ | −0.3 | −1.0 | 0.228 |
| Food intake (g/day) | 223 | 224 | 0.073 |
| Dry matter intake (g/day) | 205 | 207 | 0.073 |

¹Average for eight dogs
²Paired t-test
³Include water from deionized water, food, and SAWA
⁴% of body weight at the beginning of the test

### Example 5

### Dog Study Using Comparative Products

The comparative study was conducted in nine adult beagles using a Latin square study design. Commercial pet drinks, Waltham® Feline/Canine Veterinary Diet™ Rehydration Support™ and K9 Quencher Electrolyte Ice Cream™, were tested against drinking water. The study results are presented in Table 5.

Waltham® Feline/Canine Veterinary Diet™ Rehydration Support™ did not increase total water intake and urine production in dogs, and had poor palatability when compared with deionized water. The average intake of Waltham® Feline/Canine Veterinary Diet™ Rehydration Support™ was 212.6 g/day and the average intake of deionized water was 393.7 g/day.

The K9 Quencher Ice Cream™ significantly increased water intake and urine volume, and induced urine dilution. However, SAWA seems more efficacious in increasing total water intake and urine production in dogs. The average daily total water intake and urine production in dogs given SAWA was 930 g/day and 528 g/day, respectively, compared with 762.2 g/day and 395.4 g/day when dogs were given K9 Quencher Ice Cream™.

TABLE 5
COMPARATIVE DOG STUDY RESULTS¹

| | Water source | | | p value⁴ | |
|---|---|---|---|---|---|
| Treatments | Water | WRS² | K9-Q³ | WRS | K9-Q³ |
| Pet drink intake | | 212.6 | 444.6 | | |
| (g/day) | | | | | |
| Water intake (g/day) | 494.9 | 363.7 | 302.9 | 0.001 | 0.028 |
| Total water intake | 511.5 | 580.6 | 762.2 | 0.043 | 0.021 |
| (g/day)⁵ | | | | | |
| Total water intake | 2.7 | 2.9 | 3.8 | 0.209 | 0.025 |
| (g/g DM)⁵ | | | | | |
| Urine production | 193.6 | 224.6 | 395.4 | 0.139 | 0.043 |
| (g/day) | | | | | |
| Urine specific | 1.031 | 1.026 | 1.019 | 0.098 | 0.013 |
| gravity | | | | | |
| Urine pH | 8.09 | 8.16 | 8.17 | 0.349 | 0.386 |
| Urine phosphorus | 1301 | 1079 | 760 | 0.068 | 0.009 |
| (ppm) | | | | | |
| Urine magnesium | 90 | 70 | 45 | 0.026 | 0.005 |
| (ppm) | | | | | |
| Urine calcium | 67 | 58 | 44 | 0.166 | 0.003 |
| (ppm) | | | | | |
| Urine sodium | 1983 | 2172 | 1352 | 0.257 | 0.017 |
| (ppm) | | | | | |
| Urine potassium | 5646 | 4849 | 3611 | 0.052 | 0.013 |
| (ppm) | | | | | |
| Fecal score | 4.7 | 4.6 | 4.5 | 0.123 | 0.036 |
| Body weight | 0.73 | 4.76 | 0.41 | 0.251 | 0.633 |
| change (%)⁶ | | | | | |
| Food intake (g/day) | 209.6 | 208.2 | 215.1 | 0.401 | 0.077 |
| Dry matter intake | 192.9 | 203.6 | 200.1 | 0.025 | 0.034 |
| (g/day) | | | | | |

¹Average for nine dogs
²Waltham Feline/Canine Veterinary Diet ™ Rehydration Support ™
³K9-Quencher ™ Electrolyte Ice Cream, Randolph Ctr., VT
⁴Paired t-test, comparing with water
⁵Include water from deionized water, food, and pet drinks
⁶% of body weight at the beginning of the test

The SAWA provided improved results in comparison to commercially available pet hydration products, but did so without using supplemental sugar to enhance palatability.

1. A composition for oral consumption by a companion animal to facilitate increased total water intake and urine production as well as decreased urinary specific gravity, comprising liver digest mixed with water to form an aqueous solution or suspension, the liver digest being present in effective amounts for facilitating voluntary consumption.

2. The composition of claim 1, wherein the liver digest comprises pork liver digest.

3. The composition of claim 1, wherein the composition contains additional ingredients formulated to address the dietary requirements of a companion animal in need of increased water intake.

4. The composition of claim 1, wherein the composition contains additional ingredients formulated to meet the dietary requirements of a companion animal in need of increased water intake and having lower urinary tract disease.

5. The composition of claim 2, wherein the composition is formulated to contain additional ingredients formulated to supplement the dietary requirements of a dog or cat as the companion animal.

6. The composition of claim 1 wherein the liver digest concentration is from about 1 wt % to about 20 wt % of the composition.

7. The composition of claim 6 wherein the liver digest concentration is from about 3 wt % to about 10 wt %.

8. The composition of claim 6 wherein the liver digest is from about 3 wt % to about 7 wt %.

9. The composition of claim 6 wherein the liver digest is about 5 wt %.

10. A method of providing a companion animal the composition of claim 1, wherein the companion animal is in need of increased water intake.

11. The method of claim 10, including a step of formulating the composition with additional ingredients to supplement the diet of a dog or cat.

12. The method of claim 10, further including a step of preselecting the dog or cat by diagnosing a lower urinary tract disorder.

13. The method of claim 10, where the method is performed continuously over a period of time as a prophylactic measure to prevent a lower urinary tract disorder in the companion animal.

14. A composition for oral consumption by a companion animal comprising liver digest mixed with water to form an aqueous solution or suspension.
